(19) United States
(12) Patent Application Publication
(10) Pub. No.: US 2009/0163870 A1
(43) Pub. Date: Jun. 25, 2009

(54) TARGETING OBTURATOR

(76) Inventors: Jake Flagle, New Palistine, IN (US); Zachary R. Nicoson, Indianapolis, IN (US)

Correspondence Address: RADER, FISHMAN & GRAUER PLLC, 39533 WOODWARD AVENUE, SUITE 140, BLOOMFIELD HILLS, MI, 48304-0610, US

(21) Appl. No.: 11/961,505

(22) Filed: Dec. 20, 2007

(51) Int. Cl. A61B 17/34

(52) U.S. Cl. 604/164.01

(57) ABSTRACT

A medical targeting device includes an elongated body member extending between a distal end and a proximal end. In one embodiment, a three-dimensional targeting region is formed adjacent to the distal end such that the targeting region is formed to be substantially co-planar with an outside surface of the body member. The targeting region further includes a contrast agent. A method for using the medical targeting device is also disclosed.

### BACKGROUND

1. Technical Field

The present disclosure relates to the field of medical devices and more particularly to a targeting obturator.

2. Background

Medical procedures have advanced to stages where less invasive or minimally invasive surgeries, diagnostic procedures and exploratory procedures have become desired and demanded by patients, physicians, and various medical industry administrators. To meet these demands, improved medical devices and instrumentation have been developed, such as cannulas or micro-cannulas, medical introducers, vacuum assisted biopsy apparatus, and other endoscopic related devices.

In the field of tissue biopsy, minimally invasive biopsy devices have been developed that require only a single insertion point into a patient's body to remove one or more tissue samples. One such biopsy device incorporates a “tube-within-a-tube” design that includes an outer piercing needle having a sharpened distal end and a lateral opening that defines a tissue receiving port. An inner cutting member is slidingly received within the outer piercing needle, which serves to excise tissue that has prolapsed into the tissue receiving port. A vacuum is used to draw the excised tissue into the tissue receiving port and aspirates the excised tissue from the biopsy site once severed. Exemplary “tube-within-a-tube” biopsy devices are disclosed in pending U.S. patent application Ser. Nos. 09/707,022, 09/864,031, and 11/516,277, which are owned by the assignee of the present invention. Among other features, the exemplary biopsy devices can be used in conjunction with Magnetic Resonance Imaging (MRI). This compatibility is due to the fact that many of the components of the biopsy devices are made of materials that do not interfere with operation of MRI apparatus or are otherwise compatible therewith. It is desirable to perform biopsies in conjunction with MRI because it is a non-invasive visualization modality capable of defining the margins of a tumor.

While the exemplary MRI compatible biopsy devices have proven effective in operation, in some procedures it is desirable to create a pathway to the biopsy site for precise introduction of the biopsy device and other medical treatments into the patient. To ensure that the biopsy device is accurately placed, it is desirable to utilized a targeting obturator. However, known targeting obturators fail to indicate the potential location of a cutting portion of the biopsy device.

### SUMMARY

A medical target confirmation device, such as a targeting obturator, is disclosed. In one exemplary embodiment, the medical targeting device includes an elongated body member extending between a distal end and a proximal end and includes a targeting region formed adjacent to the distal end. The targeting region further includes a contrast agent. A method for using the medical targeting device is also disclosed.

### BRIEF DESCRIPTION OF THE DRAWINGS

Embodiments of the disclosure will now be described, by way of example, with reference to the accompanying drawings, wherein:

FIG. 1 is a side view of an embodiment of an introducer stylet;

FIG. 2 is side view of an embodiment of an outer introducer cannula;

FIG. 3 is a side view of an embodiment of a targeting obturator;

FIG. 3A is a cross-sectional view of the embodiment of the targeting obturator of FIG. 3, taken along lines 3A-3A in FIG. 3;

FIGS. 3B-G are perspective views of embodiments of a targeting obturator;

FIG. 4 is a side view of an exemplary biopsy device;

FIG. 5 is a detailed cross-sectional view of a cutting element of the biopsy device of FIG. 4;

FIG. 6 is a side view of an embodiment of an aspiration wand suitable for insertion into the outer cannula of FIG. 4; and

FIGS. 7-11 are elevational views illustrating a medical procedure.

### DETAILED DESCRIPTION

Referring now to the drawings, illustrative embodiments of the present disclosure are shown in detail. Although the drawings represent embodiments of the present disclosure, the drawings are not necessarily to scale and certain features may be exaggerated to better illustrate and explain the disclosure. Further, the embodiments set forth herein are not intended to be exhaustive or otherwise limit or restrict the disclosure to the precise forms and configurations shown in the drawings and disclosed in the following detailed description.

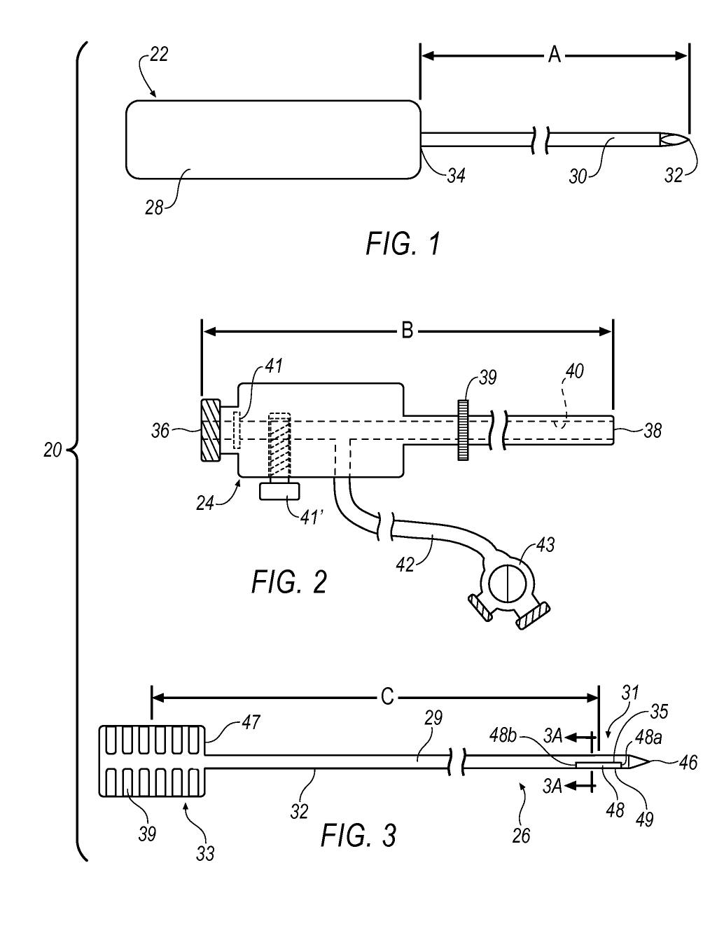

Referring to FIGS. 1-3, a medical system 20 is shown that includes an introducer stylet 22, an outer cannula 24 and a targeting obturator 26. As will be described in detail, system 20 is particularly, but not necessarily, suited for use in biopsy procedures that identify a target biopsy site using Magnetic Resonance Imaging (MRI) or comparable medical imaging modality.

An embodiment of introducer stylet 22 and outer cannula 24 may be found in co-pending U.S. patent application Ser. No. 10/649,068, the contents of which are incorporated herein by reference in its entirety.

In a particular configuration, introducer stylet 22 includes a stylet portion 30 that has a distal end with a tissue piercing tip 32, such as a trocar tip, to facilitate penetration of stylet portion 30 into a patient's tissue. In addition to, or in place of trocar tip 32, it will be appreciated that stylet portion 30 may include other devices for piercing the patient's tissue, including without limitation, devices that use a laser or radio frequencies (RF) to pierce tissue. The length of stylet portion 22 is generally denoted by the reference character “A” in FIG. 1.

Referring to the embodiment shown in FIG. 2, an embodiment of outer cannula 24 is configured to extend between an open proximal end 36 to an open distal end 38. Distal end 38 is separated from proximal end 36 by a distance “B.” Like introducer stylet portion 30, outer cannula 24 may be made from a medical grade resin or other MRI compatible material. In some configurations, proximal end 36 may include a luer-style fitting or other suitable configuration for interfacing, but not necessarily connecting, outer cannula 24 with targeting obturator 26. A depth limiting member 39, such as a rubber o-ring, may be moveably disposed on outer cannula 24 to limit the insertion depth of outer cannula 24 into the patient's body. Further, outer cannula 24 may also include an inner lumen 40 therethrough, which is open to communication with a fluid conduit 42 for supplying fluids, such as saline and anesthetics, or removing fluids, such as blood, from the patient's body. In some configurations, outer cannula 24 may include a haemostatic valve, depicted generally as element 41, or a manually operable valve 41′ that can be selectively closed to prevent the escape of fluid from proximal end 36.

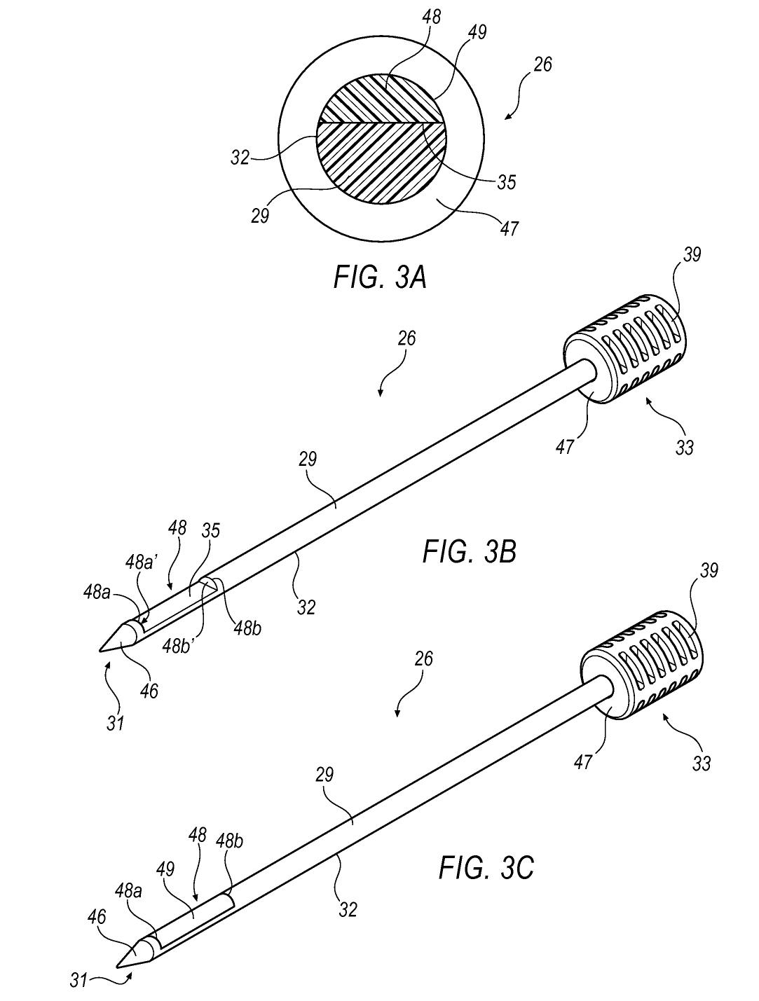

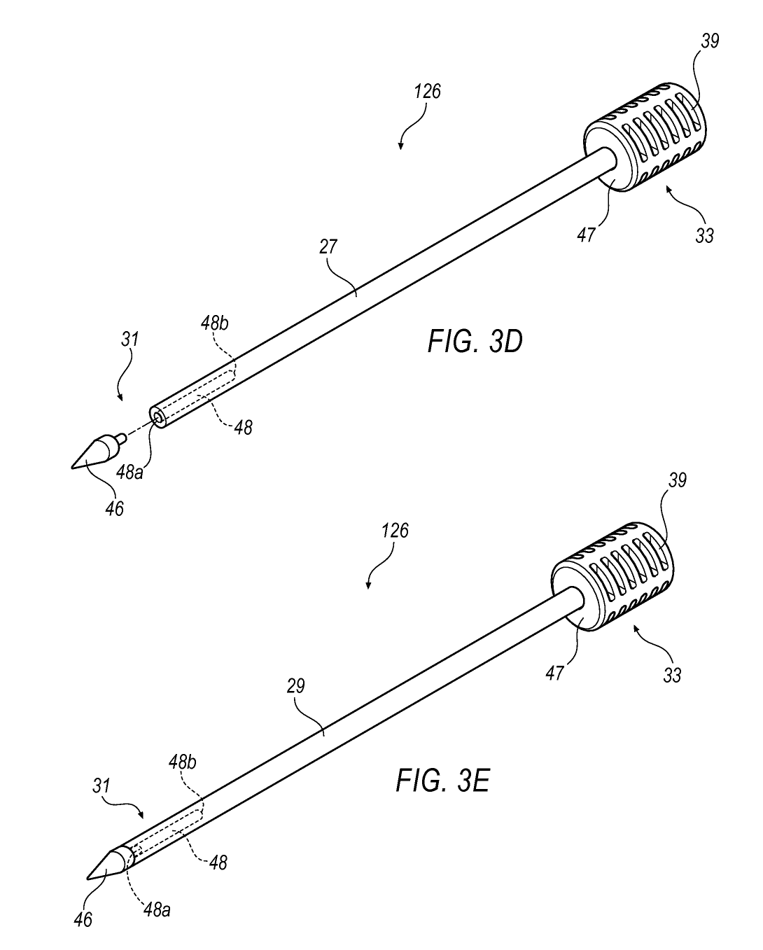

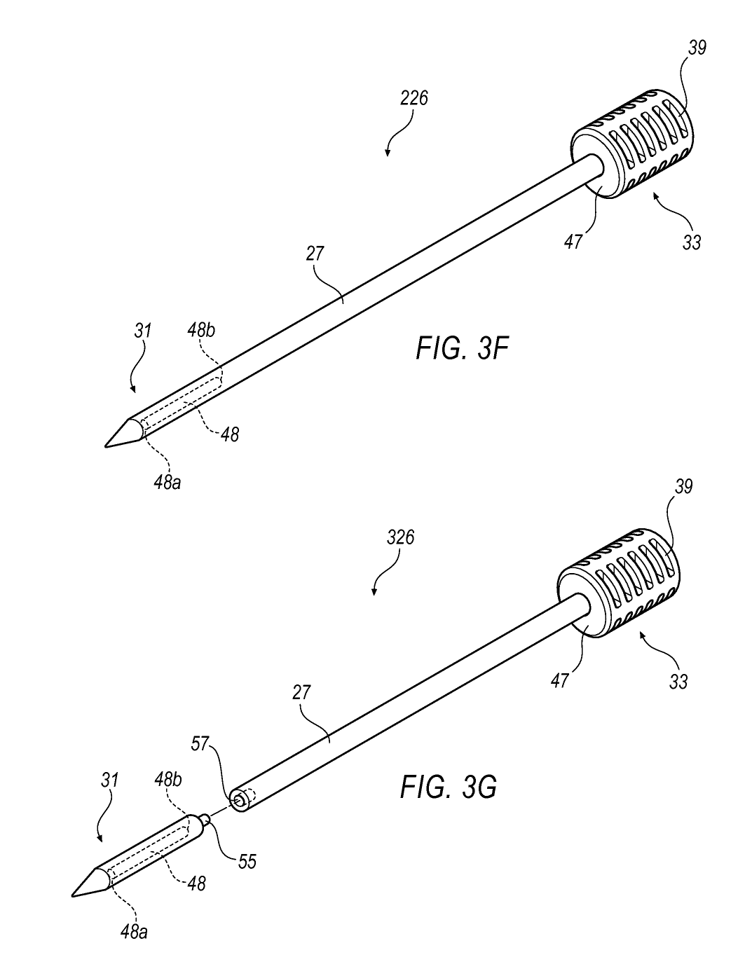

Referring to FIG. 3, an embodiment of targeting obturator 26 includes an elongated member having a distal end 31 and a proximal end 33. Targeting obturator 26 may include a body member 29 spanning between distal end 31 and proximal end 33. Body member 29 may have a generally cylindrical shape with an outer surface 32 generally sized to fit within inner lumen 40 of outer cannula 24. In one specific embodiment, body member 29, which may be made of a medical grade resin or other MRI compatible material, extends from proximal end 33 to a distal tip 46. Proximal end 33 may further include a handle 47. Body member 29 may also be made of a polymeric material such as polycarbonate. Body member 29 may have a solid composition produced from, for instance, an injection molding process, such that in one embodiment, body member 29 may be a substantially solid member. However, it is understood that body member 29 may be formed as a hollow member.

Handle 47 may abut outer cannula 24 when body member 29 is fully inserted within outer cannula 24. Handle 47 is sized so as to be at least slightly larger than an inner lumen 40 of cannula 24 such that the entire obturator 26 may not be interposed with outer cannula 24 or delivered into a patient's body. In some configurations, handle 47 may also include a luer-style fitting or other suitable feature for interfacing, but not necessarily connecting, targeting obturator 26 with outer cannula 24. Handle 47 may further include gripping members 39 to aid in the handling of targeting obturator 26. In one embodiment, gripping members 39 include a series of alternating grooves and ridges.

In one exemplary embodiment, distal tip 46 may be pointed to facilitate entry into the patient's body. However, other exemplary embodiment may include a rounded tip or even a trocar tip.

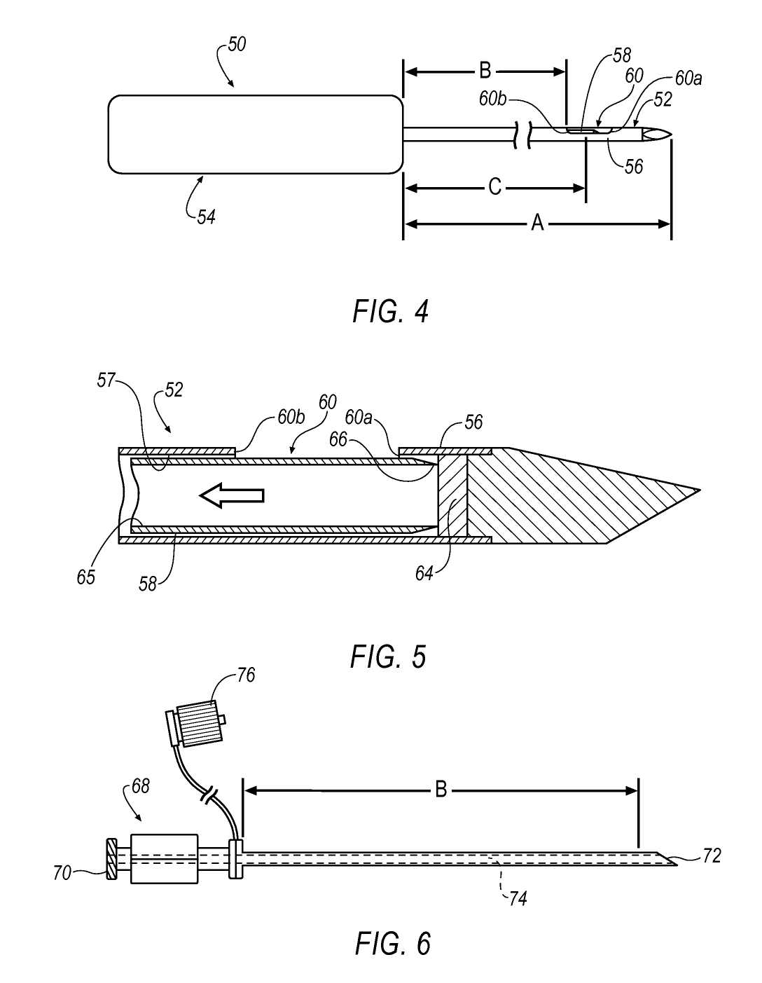

In one exemplary embodiment, a three-dimensional targeting region, or volume, 48 may be included at distal end 31 of targeting obturator 26. Targeting region 48 may be disposed, at least in part, along an outer surface 32 of the body member 29 and may extend inwardly therefrom into a portion of body member 29. Targeting region 48 generally provides an image enhancement and may further include a contrast agent. The shape of the targeting region 48 may be configured to be the approximate size of a tissue receiving opening 60 in a biopsy device 50 (see, e.g., FIG. 4). This aspect of the disclosure will be discussed in greater detail below.

In one particular configuration, the longitudinal center of targeting region 48 is provided a distance “C” from handle 47, as shown in FIG. 3. As illustrated, the distance C may be measured from the approximate center of targeting region 48 to the center of handle 47. Targeting region 48 provides a reference point in an MR image (or other suitable imaging modality) relative to a target biopsy tissue 80. Moreover, in one embodiment, the length of the targeting region as defined by edges 48a and 48b generally corresponds with the length between first and second ends 60a, 60b of the tissue-receiving opening 60 of an outer cannula 56 of the biopsy device 50.

While being able to image the longitudinal length of targeting region 48, identified by the length between edges 48a and 48b, may be advantageous, it may be also be desirable to image a three-dimensional region, or volume, approximating the tissue sample size that will be removed. As will be discussed in more detail below, tissue 80 may be coerced into tissue receiving opening 60 via vacuum suction. As a portion of the target tissue 80 is drawn into tissue receiving opening 60, an inner cannula 58 having a cutting edge disposed on a distal end thereof, moves distally across the tissue receiving opening 60, thereby severing tissue 80 and trapping a portion within an inner lumen 65 of inner cannula 58. To assist in approximating the size of the tissue samples taken by the biopsy device, the targeting region 48 may be sized and configured to be a shape generally corresponding to the size of the tissue receiving opening 60 and approximating the volume of tissue to be taken by the inner cannula 58.

An exemplary biopsy apparatus 50, which is suitable for use with medical system 20 of the present invention, is generally shown in FIG. 4 and in more detail in FIG. 5. Apparatus 50 includes a cutting element 52 sized for introduction into the patient's body and a hand piece 54. The exemplary biopsy apparatus 50 is configured as a “tube-within-a-tube” cutting device. More particularly, cutting element 52 includes an outer cannula 56 having an outer lumen 57 and an inner cannula 58 sized to fit concentrically within the outer lumen. A motor or other motion generating device is provided within hand piece 54 to rotate and/or translate inner cannula 58 within outer cannula 56. Biopsy apparatus similar to apparatus 50 can be seen by way of example in pending U.S. patent application Ser. Nos. 09/707,022 and 09/864,03, which are owned by the assignee of the present disclosure and are incorporated herein by reference in their entirety.

A particular embodiment of the working end of cutting element 52 is depicted in FIG. 5. In the illustrated embodiment, outer cannula 56 defines a tissue-receiving opening 60 (having first and second ends 60a, 60b), which communicates with outer lumen 57. The working end of cutting element 52 may further include a cutting board 64 that is disposed within outer lumen 57 at the distal end of outer cannula 56. Inner cannula 58 defines an inner lumen 65 that is hollow along its entire length to provide for aspiration of the biopsy sample (tissue). Inner cannula 58 terminates in a cutting edge 66 that may be formed by an inwardly beveled surface having a razor-sharp edge.

As discussed above, the tissue 80 may only penetrate the inner lumen 65 in the region nearest the tissue-receiving opening 60. Accordingly, targeting region 48 may be radially spaced apart or offset from a longitudinal axis of body member 29. In one exemplary embodiment, targeting region 48 may encompass an elongated arcuate volume. This arcuate volume may be bounded by a substantially rectangular base surface 35, first 48a′ and second 48b′ upright side surfaces, and an arcuate surface 49. The base surface 35 may be defined by first and second longitudinally extending edges and first and second transverse edges. First and second upright side surfaces 48a′, 48b′ may extend from said transverse edges. Additionally, the respective angles between upright side surfaces 48a′, 48b′ and base surface may exceed 90 degrees. The arcuate surface 49 may extend between said first and second upright side surfaces 48a′, 48b′ and said first and second longitudinally extending edges of base surface 35. Base surface 35 may be spaced radially from said longitudinal axis.

Targeting region 48 may include a solid contrast agent or may include a reservoir for a liquid contrast agent. In both exemplary approaches, the targeting region may be integrally formed with body member 29 of targeting obturator 26. Where body member 29 is produced by a molding process, targeting region 48 may be integrally molded into body member 29. Solid contrast agents may include a magnetic resonance imaging (MRI) identifiable metallic material, such as Inconel® 625, titanium or other material with similar magnetic characteristics. Alternatively, a liquid contrast agent may be utilized. Suitable liquid contrast agents include fluro-deoxyglucose (FDG), technicium 99, Gadolinium, and other MRI compatible contrast agents. These radioactive isotopes are visible under imaging modalities such as PET (positron emission tomography), gamma cameras, or scintimammography. The contrast agent generally shows up as a bright area under the imaging modalities. Once the obturator 26 has been placed in the body, the contrast agent will be easily visible under the imaging modalities, thereby indicating a target site where a biopsy instrument may be placed, and more specifically may correspond to the shape of the tissue sample that is to be removed.

Another embodiment of a targeting obturator 126 is shown in FIGS. 3D-3E. Targeting obturator 126 is similar to targeting obturator 26 in that is includes a body member 27 defined by a distal end 31 and a proximal end 33. Proximal end 33 may also include a handle 47 that may have gripping members 39 disposed or formed thereon.

Distal end 31 of body member 27 may be formed with a laterally extending chamber 48. Chamber 48 is generally sized and shaped to correspond to the size and shape of a tissue sample to be taken by biopsy device 50. For example, edges 48a and 48b define the margins of chamber 48. A suitable imaging material is received within chamber 48. For example, a solid contrast agent may be disposed within chamber 48, such as Inconel® 625, titanium, or other suitable material. Alternatively, a liquid contrast agent may be disposed within chamber 48. Suitable liquid contrast agents include fluro-deoxyglucose (FDG), technicium 99, Gadolinium, and other MRI compatible contrast agents.

Chamber 48 is closed off by a distal end cap 46. In one embodiment, distal end cap 46 is formed with a distal tip to facilitate entry into a patient's body. The distal tip may be pointed, rounded, or may be formed with a sharp trocar tip. Distal end cap 46 is secured to distal end of body member 27 in such a manner so as to seal off chamber 48. In one embodiment, distal end cap 46 is provided with a mounting portion that is threaded that is received within and engages corresponding threads (not shown) within chamber 48. In another embodiment, the mounting portion is press fit within chamber 48. Distal end cap may also be fixedly secured, via welding or other suitable securing operation to seal off chamber 48.

Alternative embodiments of targeting obturators 226 and 326 are depicted in FIGS. 3F-3G, respectively. Referring first to FIG. 3F, in this embodiment, the targeting region 48, defined by edges 48a and 48b, may be integrally formed with body member 27. In such an embodiment, for example, the imaging material that forms targeting region 48 may be placed in a mold cavity and the material that forms body member 27 may be molded around targeting region to provide an integral member.

In yet another alternative embodiment, referring to FIG. 3G, targeting region 48 may be integrally formed in distal end section 31 such as, for example, by placing the imaging material that forms targeting region 48 in a mold cavity and molding the distal end section 31 of targeting obturator 326 as one piece. In addition, distal end section 31 may be formed with a fitting member 55 that may be received within an opening 57 formed in separate body member 27. Other suitable mechanisms for securing distal end section 31 to body member 27 are also contemplated. For example, distal end section may be secured to body member 27 using a suitable adhesive or distal end section may be provided with an opening that receives a fitting member from body member.

Referring to FIG. 6, an aspirating wand 68 is shown that can be inserted into outer cannula 24. In an embodiment, aspirating wand 68 extends from a connecting end 70 to an insertion end 72 and includes an inner lumen 74 that extends from connecting end 70 to insertion end 72. Connecting end 70 may include a luer interface or other suitable fitting for connecting aspirating wand 68 to a vacuum source (not shown). Aspirating wand 68 may also include a cap 76 that can be placed onto connecting end 70 to inhibit fluid leakage when aspirating wand 68 is inserted into the patient. The haemostatic valve 41 in outer cannula 24 seals against aspirating wand 68, as it does against targeting obturator 26 and/or 126 and biopsy device 50, when inserted into outer cannula 24. Additionally, the outside diameter of aspirating wand 68 is less than the inside diameter of inner lumen 40 to allow saline or other fluids introduced through fluid conduit 40 to pass into the patient's body. When cap 76 is removed and aspirating wand 68 is connected to a vacuum source, fluids, such as blood and saline, can be aspirated from the biopsy site.

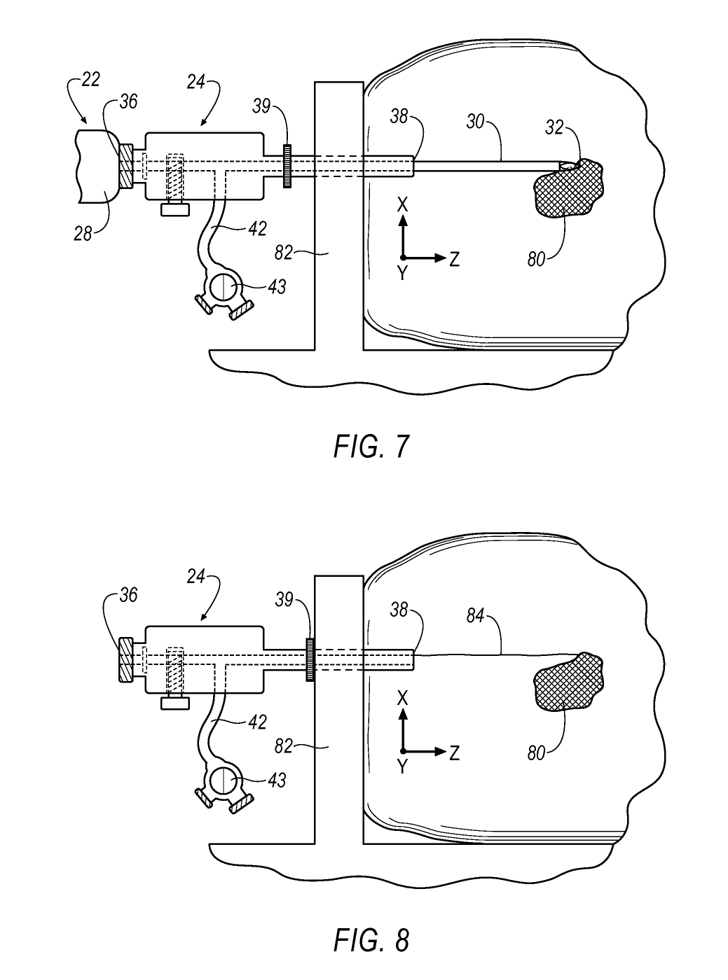

Referring to FIGS. 7-11, a medical procedure using system 20 of the present disclosure will be described. In an embodiment system 20 is employed to conduct a biopsy of a lesion within a patient's body. The target tissue or lesion to be biopsied and/or removed from the patient's body (denoted generally by mass 80 in FIG. 7) is located using a medical imaging system, such as MRI or other suitable imaging modality. A reference structure 82 may be positioned adjacent the patient to assist in locating the target tissue. The location of the target tissue 80 relative to reference structure 82 may be determined along one or more axis. In the illustrated embodiment the target tissue location relative to reference structure 82 is determined along the X and Y axes; however, the target tissue location may also be determined along all three of the X, Y and Z axes. While the described method employs a reference structure 82 to locate the target tissue, the reference structure is not necessarily required and a more “free-hand” approach may be utilized.

In an embodiment, reference structure 82 includes a support grid having a number of holes therethrough. Each hole is sized to allow passage of outer cannula 24. The hole through which outer cannula 24 is ultimately inserted is determined by the location of target tissue 80 relative to reference structure 82 along the X and Y axes. The patient and reference structure 82 are viewed using a medical imaging system, such as MRI, to determine the location of the target tissue relative to reference structure 82.

After application of anesthesia, the stylet portion of introducer stylet 22 and a portion of outer cannula 24 are inserted through the support grid and into the patient's body, creating a pathway 84 to the target tissue 80 (see, e.g., FIG. 7). In one embodiment, outer cannula 24 is sized to permit only a single instrument therewithin at a time. Thus, introducer stylet 22 is removed from the patient's body after creating the pathway, leaving behind outer cannula 24 (see, e.g., FIG. 8).

Fluids may be inserted into or removed from the patient's body through inner lumen 40 via a fluid conduit 42. These fluids may include, for example, additional anesthetics and/or saline solution to cleanse pathway 84 and remove blood. Accumulated blood and other fluids within pathway 84 may be aspirated through fluid conduit 42 or by inserting aspirating wand 68 prior to insertion of targeting obturator 26.

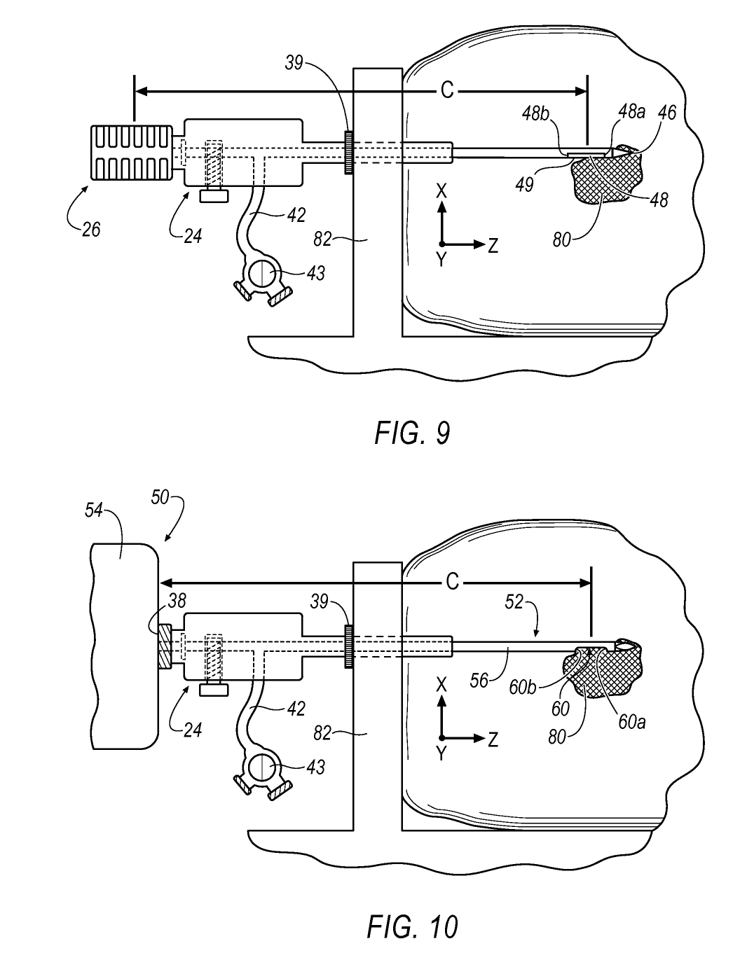

Once introducer stylet 22 is removed from outer cannula 24, targeting obturator 26 or 126 may be inserted into the patient's body through the port created by outer cannula 24 (see, e.g., FIGS. 8 and 9). With targeting obturator 26 or 126 is properly inserted into outer cannula 24, an image of the target site is again taken to determine the location of targeting region 48 in relation to the target tissue and reference structure 82. If targeting region 48 is in the desired position adjacent target tissue 80 along the Z-axis, targeting device 26 or 126 is removed from outer cannula 24. However, if targeting region 48 is not in the desired position, then the position of targeting obturator 26 and outer cannula 24 is modified along the Z-axis until the desired position is achieved.

Once the desired position is achieved, a depth limiting member 39 is moved against reference structure 82 to inhibit movement of outer cannula 24 further into the patient. When no reference structure 82 is used, depth limiting member may be moved directly against the patient's skin. Targeting obturator 26 or 126 is then removed from outer cannula 24 and biopsy device 50 is inserted into outer cannula 24 until handpiece 54 abuts proximal end 36 of outer cannula 24. In the embodiment illustrated in FIG. 10, one or more samples of target tissue 80 are removed from the patient through tissue-receiving opening 60. The correct position of tissue-receiving opening 60, as defined by first and second ends 60a, 60b, is ensured because the distance “C” between handle 47 of targeting obturator 26 and targeting region 48 (see, e.g., FIGS. 3 and 9), is approximately equal to the distance between the center of tissue receiving opening 60 and handpiece 54 of biopsy device 50.

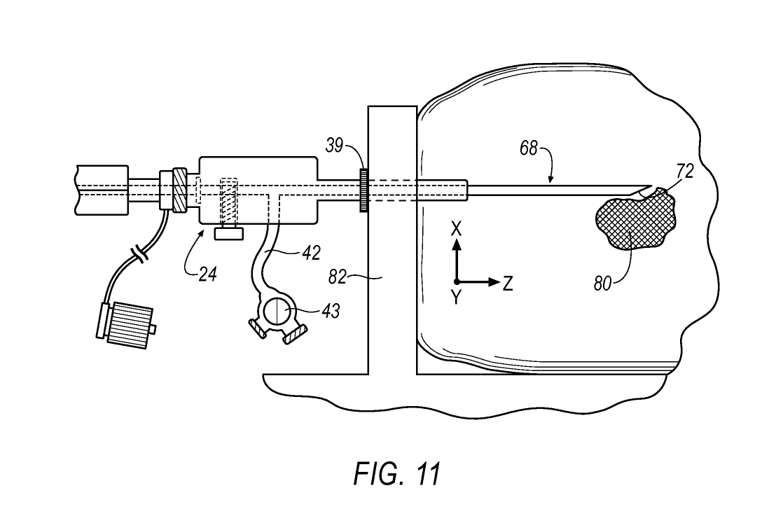

After completion of the biopsy, the biopsy site can be aspirated using aspirating wand 68 (see, e.g., FIG. 11). During or after aspiration, a final image of the biopsy site can be taken to confirm removal of the target tissue. Finally, an identifiable marker, such as a collagen plug, or other medical treatment can be inserted into the biopsy site through outer cannula 24.

Among other features, the medical system of the present invention localizes the target biopsy site in a manner that allows confirmation of the target biopsy site under MRI or other visualization modality, and allows positioning of a biopsy device to ensure the cutting element of the biopsy device can be accurately placed at the target biopsy site. The medical system of the present invention also facilitates the introduction and removal of fluids from the target site, including without limitation, anesthesia and blood, but minimizes the exposure of the fluids to the adjacent equipment and medical staff. In addition to allowing the medical staff to identify the presence of significant bleeding and to introduce a biopsy device into the patient, the medical system provides access to the target site to introduce a medical treatment, such as a site marker, tamponade or other haemostatic agent, after removal of the tissue.

The present disclosure has been particularly shown and described with reference to the foregoing embodiments, which are merely illustrative out of the disclosure. It should be understood by those skilled in the art that various alternatives to the embodiments of the disclosure described herein may be employed in practicing the disclosure without departing from the spirit and scope of the disclosure as defined in the following claims. It is intended that the following claims define the scope of the invention and that the method and apparatus within the scope of these claims and their equivalents be covered thereby. This description of the disclosure should be understood to include all novel and non-obvious combinations of elements described herein, and claims may be presented in this or a later application to any novel and non-obvious combination of these elements. Moreover, the foregoing embodiments are illustrative, and no single feature or element is essential to all possible combinations that may be claimed in this or a later application.

What is claimed is:

1. A medical targeting device, comprising:
an elongated body member extending between a distal end and a proximal end;
a three-dimensional targeting region formed adjacent to said distal end, wherein said targeting region is formed so as to be substantially coplanar with an outside surface of said body member and having a portion that extends inwardly therefrom into a portion of said body member; and
wherein said targeting region includes a contrast agent.

2. The device of claim 1, wherein said body member further comprises a generally centrally located longitudinal axis extending therethrough and wherein said targeting region is radially offset from said axis.

3. The device of claim 1, wherein said body member is generally cylindrical and said targeting region includes an elongated arcuate volume.

4. The device of claim 3, wherein said arcuate volume includes:
a substantially rectangular base portion defined by first and second generally longitudinally extending edges and first and second generally transverse edges;
first and second upright side surfaces extending from said transverse edges;
a generally arcuate surface extending between said first and second upright side surfaces and said first and second longitudinally extending edges.

5. The device of claim 4, wherein said body member further comprises a generally centrally located longitudinal axis extending therethrough and wherein said generally rectangular base surface is spaced radially from said longitudinal axis.

6. The device of claim 1, wherein said contrast agent is metallic.

7. The device of claim 1, wherein said targeting region is a liquid contrast agent.

8. The device of claim 1, wherein said body member is solid.

9. The device of claim 1, wherein said body member is a polymeric material.

10. The device of claim 1, wherein said targeting region is integrally formed with said body member.

11. The device of claim 10, wherein said targeting region is integrally molded into said body member.

12. A medical targeting device, comprising:
an elongated body member extending between a distal end and a proximal end;
a laterally extending and defined chamber positioned adjacent the distal end and extending inwardly therefrom a predetermined distance; and
a contrast agent positioned within said chamber.

13. The medical targeting device of claim 12, wherein said chamber is closed off at said distal end by a distal end cap.

14. The medical targeting device of claim 13, wherein said distal end cap is secured to said distal end so as to seal said chamber.

15. The medical targeting device of claim 13, wherein said distal end cap further includes a mounting portion that is at least partially received within said chamber.

16. The medical targeting device of claim 12, wherein said body member is a generally solid member.

17. The medical targeting device of claim 12, wherein said contrast agent is molded into said chamber.

18. A medical targeting device, comprising:
A elongated body member extending between a first end and a second end; and
a targeting region comprised of a contrast agent integrally formed with at least a portion of the targeting device, adjacent a distal end of the targeting device and extending inwardly therefrom a predetermined distance.

19. The medical targeting device of claim 18, wherein the elongated body member is solid and the targeting region is integrally formed in the second end of elongated body member so as to form a unitary member.

20. The medical targeting device of claim 18, further comprising a distal end section, wherein the targeting region is integrally formed with the distal end section and wherein the distal end section operatively connects to the first end of the elongated body member.
